(19) United States
(12) Patent Application Publication
(10) Pub. No.: US 2016/0345995 A1
(43) Pub. Date: Dec. 1, 2016

(54) TREATMENT DEVICE

(71) Applicant: OLYMPUS CORPORATION, Tokyo (JP)

(72) Inventor: Yusuke TAKEI, Hino-shi (JP)

(73) Assignee: OLYMPUS CORPORATION, Tokyo (JP)

(21) Appl. No.: 15/231,366

(22) Filed: Aug. 8, 2016

Related U.S. Application Data
(63) Continuation of application No. PCT/JP2015/053268, filed on Feb. 5, 2015.

(30) Foreign Application Priority Data
Feb. 12, 2014 (JP) 2014-024755

(51) Int. Cl. A61B 17/29

(52) U.S. Cl. CPC A61B 17/29; A61B 2017/00393

(57) ABSTRACT

A treatment device includes an insertion portion, an end effector, a grasp portion, a transmitting member, a first locking portion, a second locking portion, and an operational body. The operational body is configured to switch between a first position where the transmitting member is moved forward along the longitudinal axis of the insertion portion to keep the end effector in a first state to extend along the longitudinal axis to the distal end of the insertion portion along the longitudinal axis, and a second position where the transmitting member is moved backward along the longitudinal axis of the insertion portion to keep the end effector in a second state to be rotated relative to the longitudinal axis with respect to the distal end of the insertion portion.

### CROSS-REFERENCE TO RELATED APPLICATIONS

This application is a Continuation Application of PCT Application No. PCT/JP2015/053268, filed Feb. 5, 2015 and based upon and claiming the benefit of priority from prior Japanese Patent Application No. 2014-024755, filed Feb. 12, 2014, the entire contents of all of which are incorporated herein by reference.

### BACKGROUND OF THE INVENTION

1. Field of the Invention

This invention relates to a treatment device to treat living tissues.

2. Description of the Related Art

For example, Jpn. Pat. Appln. KOKAI Publication No. 2012-35065 discloses a treatment device having an end effector which is rotatable relative to the distal end of a sheath. This treatment device locks and keeps the end effector rotated relative to the sheath so that a fixture may be easily attached to the end of the end effector. After the fixture is attached, the end effector is unlocked and thus held straight relative to the distal end of the sheath.

### BRIEF SUMMARY OF THE INVENTION

According to one aspect of the present invention, a treatment device includes an insertion portion which includes a distal end, a proximal end, and a longitudinal axis defined by the distal end and the proximal end; an end effector which is rotatably provided at the distal end of the insertion portion; a grasp portion which is provided at the proximal end of the insertion portion and which is grasped by a user; a transmitting member which moves back and forth along a longitudinal axis of the insertion portion to transmit a drive force to rotate the end effector; a first locking portion which is provided on the proximal side of the transmitting member; a second locking portion which is provided on the proximal side of the insertion portion and which is configured to engage with the first locking portion; and an operational body which activates the first locking portion relative to the second locking portion, which is configured to switch between a first position where the transmitting member is moved forward along the longitudinal axis of the insertion portion to keep the end effector in a first state to extend along the longitudinal axis relative to the distal end of the insertion portion along the longitudinal axis, and a second position where the transmitting member is moved backward along the longitudinal axis of the insertion portion to keep the end effector in a second state to be rotated relative to the longitudinal axis with respect to the distal end of the insertion portion.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1A is a schematic partly sectional view showing a treatment device according to first and second embodiments;

FIG. 1B is a schematic diagram showing a position holding mechanism to hold the position of a transmitting member relative to a grasp portion of the treatment device according to the first and second embodiments;

FIG. 1C is a schematic diagram showing the position holding mechanism to hold the position of the transmitting member relative to the grasp portion of the treatment device according to the first and second embodiments;

FIG. 2 is a schematic partly sectional perspective view showing the position holding mechanism to hold the position of the transmitting member relative to the grasp portion of the treatment device according to the first and second embodiments;

FIG. 3A is a schematic diagram showing a first locking portion which is disposed on the proximal side of the transmitting member in relation to a second locking portion which is disposed in the grasp portion of the treatment device according to the first embodiment;

FIG. 3B is a schematic sectional view taken along the line 3B-3B in FIG. 3A, showing the first locking portion which is disposed on the proximal side of the transmitting member in relation to the second locking portion which is disposed in the grasp portion of the treatment device according to the first embodiment;

FIG. 4A is a schematic partly sectional view taken along the line 4A-4A in FIG. 4B, showing the position of an engagement pawl of the first locking portion which is disposed on the proximal side of the transmitting member in relation to an engagement groove of the second locking portion which is disposed in the grasp portion of the treatment device according to the first embodiment;

FIG. 4B is a schematic sectional view taken along the line 4B-4B in FIG. 4A, showing the position of the engagement pawl of the first locking portion which is disposed on the proximal side of the transmitting member in relation to the engagement groove of the second locking portion which is disposed in the grasp portion of the treatment device according to the first embodiment;

FIG. 5A is a schematic partly sectional view taken along the line 5A-5A in FIG. 5B, showing the position of the engagement pawl of the first locking portion which is disposed on the proximal side of the transmitting member in relation to the engagement groove of the second locking portion which is disposed in the grasp portion of the treatment device according to the first embodiment;

FIG. 5B is a schematic sectional view taken along the line 5B-5B in FIG. 5A, showing the position of the engagement pawl of the first locking portion which is disposed on the proximal side of the transmitting member in relation to the engagement groove of the second locking portion which is disposed in the grasp portion of the treatment device according to the first embodiment;

FIG. 6A is a schematic partly sectional view taken along the line 6A-6A in FIG. 6B, showing the position of the engagement pawl of the first locking portion which is disposed on the proximal side of the transmitting member in relation to the engagement groove of the second locking portion which is disposed in the grasp portion of the treatment device according to the first embodiment;

FIG. 6B is a schematic sectional view taken along the line 6B-6B in FIG. 6A, showing the position of the engagement pawl of the first locking portion which is disposed on the proximal side of the transmitting member in relation to the engagement groove of the second locking portion which is disposed in the grasp portion of the treatment device according to the first embodiment;

FIG. 7A is a schematic partly sectional view taken along the line 7A-7A in FIG. 7B, showing the position of the engagement pawl of the first locking portion which is disposed on the proximal side of the transmitting member in relation to the engagement groove of the second locking portion which is disposed in the grasp portion of the treatment device according to the first embodiment;

FIG. 7B is a schematic sectional view taken along the line 7B-7B in FIG. 7A, showing the position of the engagement pawl of the first locking portion which is disposed on the proximal side of the transmitting member in relation to the engagement groove of the second locking portion which is disposed in the grasp portion of the treatment device according to the first embodiment;

FIG. 8A is a schematic partly sectional view taken along the line 8A-8A in FIG. 8B, showing the position of the engagement pawl of the first locking portion which is disposed on the proximal side of the transmitting member in relation to the engagement groove of the second locking portion which is disposed in the grasp portion of the treatment device according to the first embodiment;

FIG. 8B is a schematic sectional view taken along the line 8B-8B in FIG. 8A, showing the position of the engagement pawl of the first locking portion which is disposed on the proximal side of the transmitting member in relation to the engagement groove of the second locking portion which is disposed in the grasp portion of the treatment device according to the first embodiment;

FIG. 9A is a schematic diagram showing a rotating cam type position holding mechanism of the treatment device according to the first embodiment, and showing a state in which a cam provided in the transmitting member is disengaged from a recess formed in the outer circumferential surface of a grasp side cylindrical portion of the grasp portion;

FIG. 9B is a schematic diagram showing the rotating cam type position holding mechanism of the treatment device according to the first embodiment, and showing a state in which the cam provided in the transmitting member is engaged with the recess formed in the outer circumferential surface of the grasp side cylindrical portion of the grasp portion;

FIG. 10 is a schematic diagram showing a ratchet cam type position holding mechanism of the treatment device according to the first embodiment, and showing a state in which the position of an operational body is moved relative to a pawl formed in the outer circumferential surface of the grasp side cylindrical portion of the grasp portion;

FIG. 11A is a schematic diagram showing the ratchet cam type position holding mechanism of the treatment device according to the first embodiment, and showing a state in which a movable body and an elastic member are started to be pressed by the operational body from a state in which the movable body and the elastic member are supported on a tubular body;

FIG. 11B is a schematic diagram showing the ratchet cam type position holding mechanism of the treatment device according to the first embodiment, and showing a state in which the movable body and the elastic member are pressed by the operational body so that a protrusion of the movable body is engaged with a first cutout in the inner circumferential surface of the tubular body;

FIG. 11C is a schematic diagram showing the ratchet cam type position holding mechanism of the treatment device according to the first embodiment, and showing a state in which the movable body and the elastic member are again pressed by the operational body so that the protrusion of the movable body is disengaged from the first cutout in the inner circumferential surface of the tubular body, and the protrusion is moving along an angle portion of the tubular body;

FIG. 11D is a schematic diagram showing the ratchet cam type position holding mechanism of the treatment device according to the first embodiment, and showing a state in which the protrusion of the movable body is engaged with a second cutout of the tubular body;

FIG. 12A is a schematic longitudinal sectional view showing a connection state between the proximal end of an end effector of the treatment device according to the second embodiment and the sheath and the distal end of the transmitting member, and showing a state in which the end effector is straight along the longitudinal axis of the sheath;

FIG. 12B is a schematic longitudinal sectional view showing a connection state between the proximal end of the end effector of the treatment device according to the second embodiment and the sheath and the distal end of the transmitting member, and showing a state in which the transmitting member has retreated relative to the sheath along the longitudinal axis;

FIG. 12C is a schematic longitudinal sectional view showing a connection state between the proximal end of the end effector of the treatment device according to the second embodiment and the sheath and the distal end of the transmitting member, and showing a state in which the transmitting member retreats relative to the sheath along the longitudinal axis so that the end effector starts to rotate in a direction to deviate from the longitudinal axis;

FIG. 12D is a schematic longitudinal sectional view showing a connection state between the proximal end of the end effector of the treatment device according to the second embodiment and the sheath and the distal end of the transmitting member, and showing a state in which the transmitting member retreats relative to the sheath along the longitudinal axis so that the end effector is held rotated in the direction to deviate from the longitudinal axis;

FIG. 13 is a schematic longitudinal sectional view showing a connection state between the proximal end of the end effector of the treatment device according to a first modification of the second embodiment and the sheath and the distal end of the transmitting member;

FIG. 14 is a schematic longitudinal sectional view showing a connection state between the proximal end of the end effector of the treatment device according to a second modification of the second embodiment and the sheath and the distal end of the transmitting member; and

FIG. 15 is a schematic diagram showing a mechanism which adjusts the rotation amount of the end effector of the treatment device according to a third modification of the second embodiment.

### DETAILED DESCRIPTION OF THE INVENTION

Hereinafter, embodiments of this invention will be described with reference to the drawings.

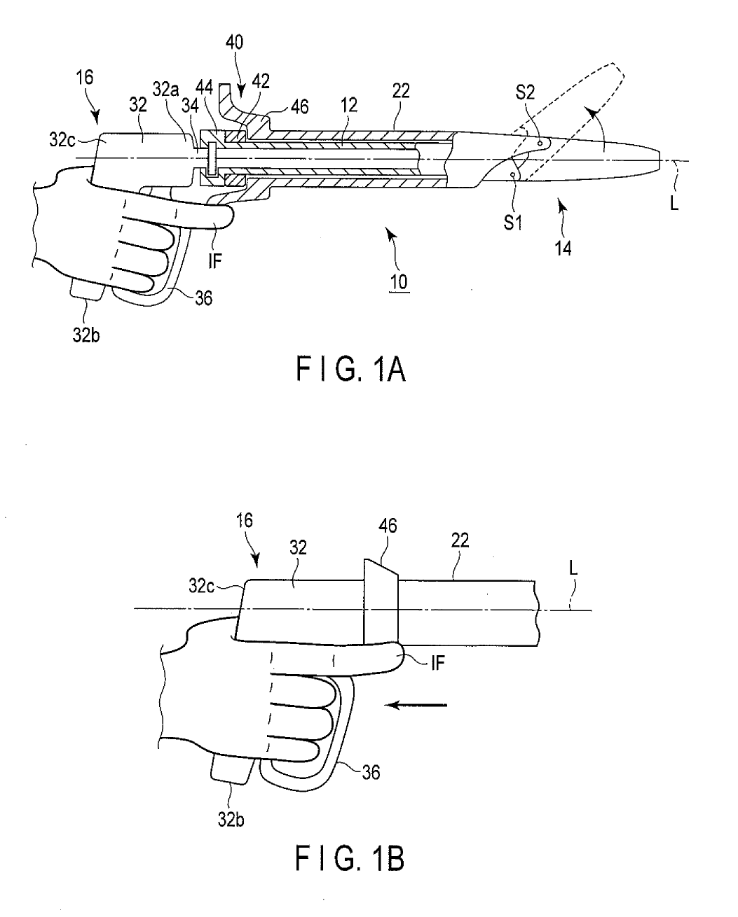

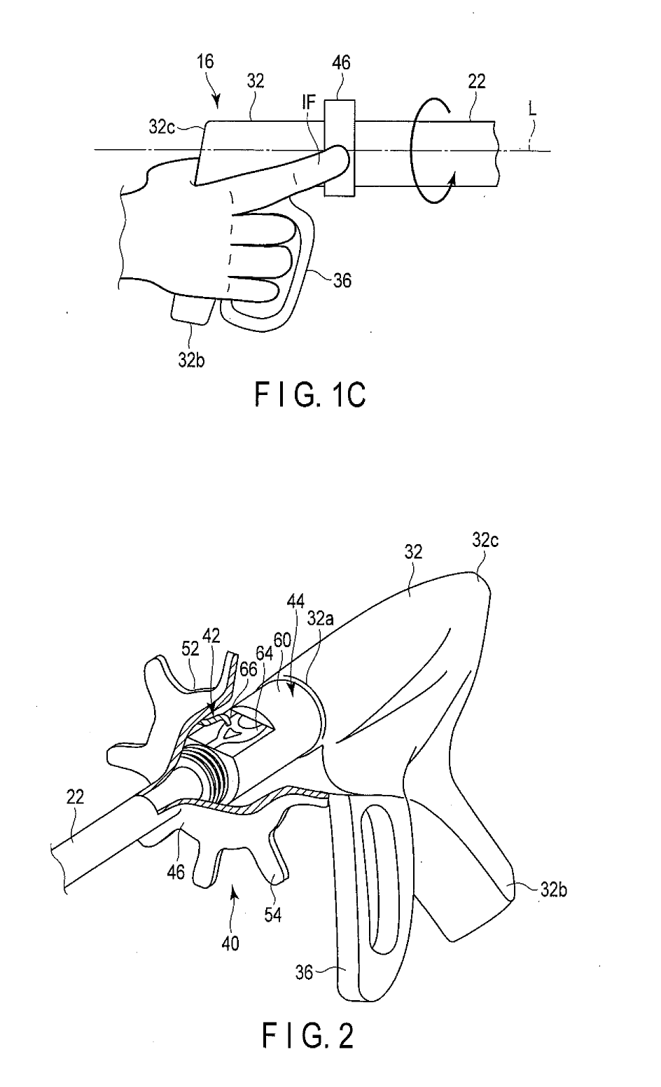

The first embodiment is described with reference to FIG. 1A to FIG. 8B.

As shown in FIG. 1A, a treatment device 10 according to this embodiment has an elongated sheath (inner tube) 12, an end effector 14, and a grasp portion 16. The sheath 12 functions as an insertion portion to be inserted into, for example, a body cavity together with the end effector 14.

The sheath 12 has a distal end, a proximal end, and a longitudinal axis (central axis) L defined by the distal end and the proximal end. Thus, a front (distal side) and a back (proximal side) are defined along the longitudinal axis L. The sheath 12 is cylindrically formed in this embodiment. The sheath 12 is formed as an inner tube which is disposed inside a later-described transmitting member 22 as an outer tube. Thus, the longitudinal axis L is also the central axis of the sheath 12 and the transmitting member 22. The proximal end of the end effector 14 is coupled to the distal end of the sheath 12 rotatably on a first rotation shaft S1. The first rotation shaft S1 intersects at right angles with the longitudinal axis (central axis) L, and is located apart from the longitudinal axis (central axis) L.

The end effector 14 is, for example, openable and closable by a known mechanism. Thus, the end effector 14 can hold and push away living tissues. The end effector 14 is rotatably provided at the distal end of the sheath 12.

The transmitting member (outer tube) 22 which can move back and forth along the longitudinal axis L and which can transmit a drive force to rotate the end effector 14 to the distal end of the sheath 12 is disposed in the sheath 12. The transmitting member 22 is cylindrically formed as an outer tube outside the sheath 12 in this embodiment. The distal end of the transmitting member 22 is coupled so that the proximal end of the end effector 14 is rotatable on a second rotation shaft S2.

The end effector 14 according to this embodiment is formed to be rotatable only in one direction from a straight state. The second rotation shaft S2 is located apart from the longitudinal axis (central axis) L and is parallel to the first rotation shaft S1. That is, the second rotation shaft S2 intersects at right angles with the longitudinal axis (central axis) L. The second rotation shaft S2 is located opposite to the first rotation shaft S1 across the longitudinal axis (central axis) L.

If the transmitting member 22 is moved forward relative to the sheath 12 along the longitudinal axis L, the end effector 14 is disposed at a position (first position) where the end effector 14 is straight along the longitudinal axis L. If the transmitting member 22 is moved backward relative to the sheath 12 along the longitudinal axis L, the end effector 14 is disposed at a position (second position) where the end effector 14 is rotated to a predetermined rotation angle relative to the longitudinal axis L.

As shown in FIG. 1A to FIG. 1C, the grasp portion 16 is provided at the proximal end of the sheath 12, and is grasped by a user. The grasp portion 16 has a main body 32 which is substantially L-shaped like a pistol to be grasped in this embodiment, a grasp side cylindrical portion 34, and an open-close knob 36.

The main body 32 has one end 32a, the other end 32b, and a bent portion 32c. When grasping the grasp portion 16, the user of the treatment device 10 grasps a part ranging from the bent portion 32c to the other end 32b, and then extends, for example, the index to little fingers toward the one end 32a of the grasp portion 16.

The open-close knob 36 is disposed in the main body 32 of the grasp portion 16 when the end effector 14 has a pair of openable and closable jaws (not shown). The open-close knob 36 is disposed from the bent portion 32c to the one end 32a of the main body 32. The open-close knob 36 can move closer to or away from the other end 32b of the main body 32. The open-close knob 36 is coupled to the end effector 14 by, for example, an unshown wire which is inserted through the sheath 12. For example, when the open-close knob 36 is moved closer to the other end 32b of the main body 32, the wire is pulled so that the pair of jaws are relatively closed. For example, when the open-close knob 36 is moved away from the other end 32b of the main body 32, the wire is pushed out so that the pair of jaws are relatively opened. It is possible to relatively open and close the pair of jaws by operating the open-close knob 36 in this way.

The grasp side cylindrical portion 34 is disposed at the one end 32a of the main body 32. The proximal end of the sheath 12 is connected rotatably relative to the grasp side cylindrical portion 34 of the grasp portion 16 around the longitudinal axis L. A later-described base 60 of a second locking portion 44 is also rotatable relative to the grasp side cylindrical portion 34 around the longitudinal axis L.

As shown in FIG. 1A and FIG. 2 to FIG. 8B, a first locking portion 42 is provided on the proximal side of the transmitting member 22. The second locking portion 44 which can engage with the first locking portion 42 is provided in the outer circumferential surface of the grasp side cylindrical portion 34 of the grasp portion 16 or in the outer circumferential surface of the proximal end of the sheath 12. That is, the second locking portion 44 which can engage with the first locking portion 42 is provided on the proximal side of the sheath 12. In the explanation according to this embodiment, the sheath 12 and the transmitting member 22 rotate around the longitudinal axis L, so that the second locking portion 44 is provided in the outer circumferential surface of the proximal end of the sheath 12. If the sheath 12 and the transmitting member 22 do not need to rotate around the longitudinal axis L, the second locking portion 44 may be provided in the outer circumferential surface of the grasp side cylindrical portion 34 of the grasp portion 16. An operational body 46 which activates the first locking portion 42 relative to the second locking portion 44 is formed on the proximal side of the transmitting member 22. The first locking portion 42, the second locking portion 44, and the operational body 46 form a position holding mechanism (alternate mechanism) 40 by which the first locking portion 42 and the second locking portion 44 hold positions relative to each other. The position holding mechanism 40 is provided between the transmitting member 22 and the grasp portion 16.

As shown in FIG. 2 and FIG. 4A to FIG. 8B, the operational body 46 is integrated with the proximal end of the transmitting member 22 in this embodiment. The operational body 46 has a cylindrical portion 52, and a substantially disc-shaped extension 54 which is disposed on the outer circumference of the cylindrical portion 52 and which extends outward in the diametrical direction.

The cylindrical portion 52 is formed so that the later-described base 60 of the second locking portion 44 is disposed inside the cylindrical portion 52.

As described above, the extension 54 is located so that the user of the treatment device 10 can move the extension 54 along the longitudinal axis L by putting an index finger IF on the extension 54 when the user grasps the part of the grasp portion 16 ranging from the bent portion 32c to the other end 32b and then extends the index to little fingers toward the one end 32a of the grasp portion 16. Thus, the index finger IF is put on the extension 54 to move the transmitting member 22 along the direction of the longitudinal axis L. A step 56 is formed in the operational body 46 between the operational body 46 and the proximal end of the transmitting member 22, and the distal end of a later-described urging member 58 is supported by the step 56.

The urging member 58, for example, a coil spring is disposed between the step 56 of the operational body 46 and the distal end of the later-described cylindrical base 60. The urging member 58 urges the transmitting member 22 forward along the direction of the longitudinal axis L.

As shown in FIG. 2 to FIG. 8B, the position holding mechanism 40 is formed as a heart cam type in this embodiment.

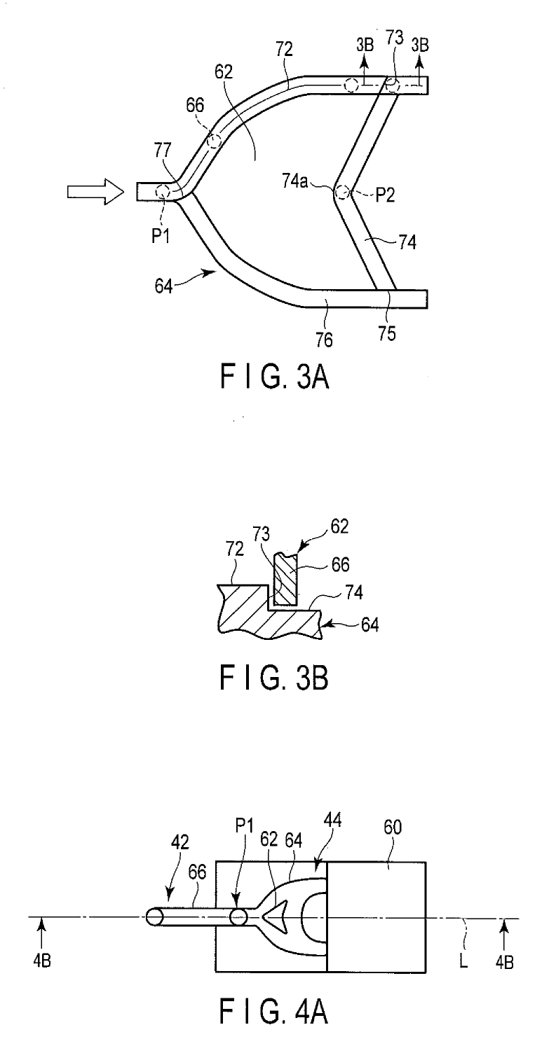

As shown in FIG. 3A and FIG. 3B, the second locking portion 44 has the cylindrical base 60, a heart-shaped island 62 formed in the outer circumferential surface of the base 60, and a locking groove 64 formed around the island 62. The first locking portion 42 has an engagement pawl 66 which is movable along the locking groove 64 and which is elastically deformable. The engagement pawl 66 is urged to be movable along the bottom surface of the locking groove 64. The engagement pawl 66 is movable between an unlock position (first position) P1 and a lock position (second position) P2 of the locking groove 64 that will be described later, and is held at the unlock position P1 and the lock position P2. That is, if the engagement pawl 66 of the first locking portion 42 traces the locking groove 64 of the second locking portion 44, the engagement pawl 66 moves from the unlock position P1 to the lock position P2 of the locking groove 64, or from the lock position P2 to the unlock position P1. The engagement pawl 66 of the position holding mechanism 40 then performs an alternating action along the locking groove 64 by the urging member 58.

The locking groove 64 has a first groove portion 72, a second groove portion 74, and a third groove portion 76. The locking groove 64 forms the aforementioned island 62 inside by the first to third groove portions 72, 74, and 76. The boundary between the first groove portion 72 and the second groove portion 74 has a step 73 in which the first groove portion 72 is formed high and the second groove portion 74 is formed low. The boundary between the second groove portion 74 and the third groove portion 76 has a step 75 in which the second groove portion 74 is formed high and the third groove portion 76 is formed low. The boundary between the third groove portion 76 and the first groove portion 72 has a step 77 in which the third groove portion 76 is formed high and the first groove portion 72 is formed low.

The first groove portion 72 is formed higher in the vicinity of the boundary between the first groove portion 72 and the second groove portion 74 than in the vicinity of the boundary between the first groove portion 72 and the third groove portion 76. The second groove portion 74 is formed lower in the vicinity of the boundary between the second groove portion 74 and the first groove portion 72 than in the vicinity of the boundary between the second groove portion 74 and the third groove portion 76. The third groove portion 76 is formed higher in the vicinity of the boundary between the third groove portion 76 and the first groove portion 72 than in the vicinity of the boundary between the third groove portion 76 and the second groove portion 74. That is, each of the first groove portion 72 to the third groove portion 76 is formed as an inclined surface.

The first groove portion 72 has the unlock position (first position) P1 where the engagement pawl 66 is disengaged in the vicinity of the boundary between the first groove portion 72 and the third groove portion 76. The second groove portion 74 is substantially V-shaped, that is, has a bent portion 74a, and the bent portion 74a functions as the lock position (second position) P2 of the engagement pawl 66.

At the unlock position (first position) P1, the transmitting member 22 is moved forward along the longitudinal axis L of the sheath 12 by the operational body 46. That is, the transmitting member 22 is moved to a front position. In this instance, the end effector 14 is kept straight along the longitudinal axis L relative to the distal end of the sheath 12. At the lock position (second position) P2, the transmitting member 22 is moved backward along the longitudinal axis L of the sheath 12 by the operational body 46. That is, the transmitting member 22 is moved backward and then slightly returned forward and is thereby moved to a back position located backward of the front position. In this instance, the end effector 14 is kept rotated relative to the longitudinal axis L with respect to the distal end of the sheath 12. These positions are switched by the operation of the operational body 46. That is, the operational body 46 of the position holding mechanism 40 can be switched between the unlock position (first position) P1 where the transmitting member 22 is kept relatively moved forward along the longitudinal axis L to keep the end effector 14 in a first state to be straight along the longitudinal axis L, and the lock position (second position) P2 where the transmitting member 22 is kept relatively moved backward along the longitudinal axis L to keep the end effector 14 in a second state to be rotated relative to the longitudinal axis L at the distal end of the sheath 12. The position holding mechanism 40 is switched between the first position and the second position by the alternating action.

Functions according to this embodiment are described with reference to FIG. 1A to FIG. 1c and FIG. 4A to FIG. 8B.

As shown in FIG. 1A, the user grasps the position of the grasp portion 16 ranging from the bent portion 32c to the other end 32b and then extends the index finger IF toward the operational body 46 to put the index finger IF on the extension 54. The user inserts the end effector 14, the distal end of the sheath 12, and the distal end of the transmitting member 22 into, for example, a body cavity.

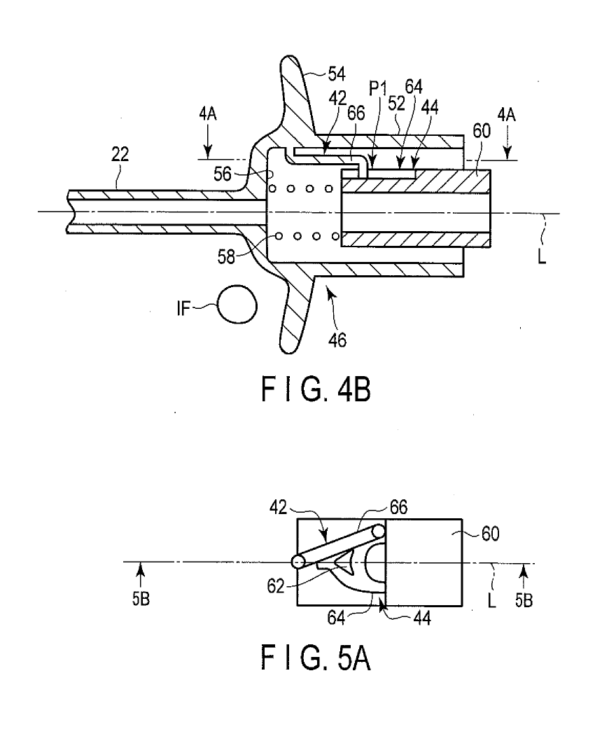

As shown in FIG. 4A and FIG. 4B, in a state in which the operational body 46 is relatively moved forward along the longitudinal axis L to keep the end effector 14 straight along the longitudinal axis L, the engagement pawl 66 of the first locking portion 42 is disposed at the position (unlock position P1) of the first groove portion 72 in the boundary between the first groove portion 72 and the third groove portion 76 of the second locking portion 44.

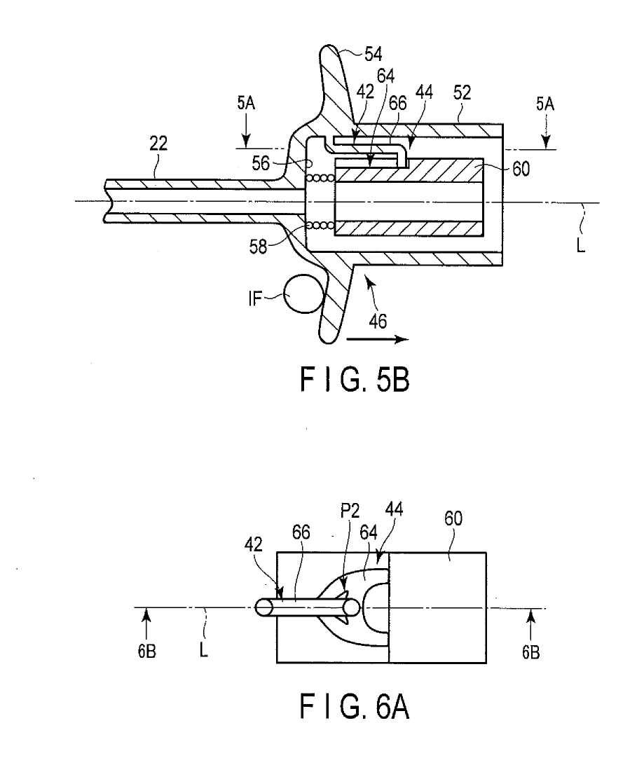

From this state, the extension 54 of the operational body 46 is operated backward on the longitudinal axis L. That is, the first locking portion 42 is moved relative to the second locking portion 44. The engagement pawl 66 of the first locking portion 42 cannot move from the first groove portion 72 of the second locking portion 44 to the third groove portion 76 due to the step 77, and moves toward the boundary between the first groove portion 72 and the second groove portion 74 from the unlock position P1 as shown in FIG. 5A and FIG. 5B.

In this instance, the transmitting member 22 is pulled backward by a component in a direction along the longitudinal axis L in the distance between the unlock position P1 and the boundary between the first groove portion 72 and the second groove portion 74. That is, the transmitting member 22 is pulled backward relative to the sheath 12. Thus, the end effector 14 is pulled on the second rotation shaft S2, and the end effector 14 rotates around the first rotation shaft S1. In this instance, the living tissues can be, for example, pushed away.

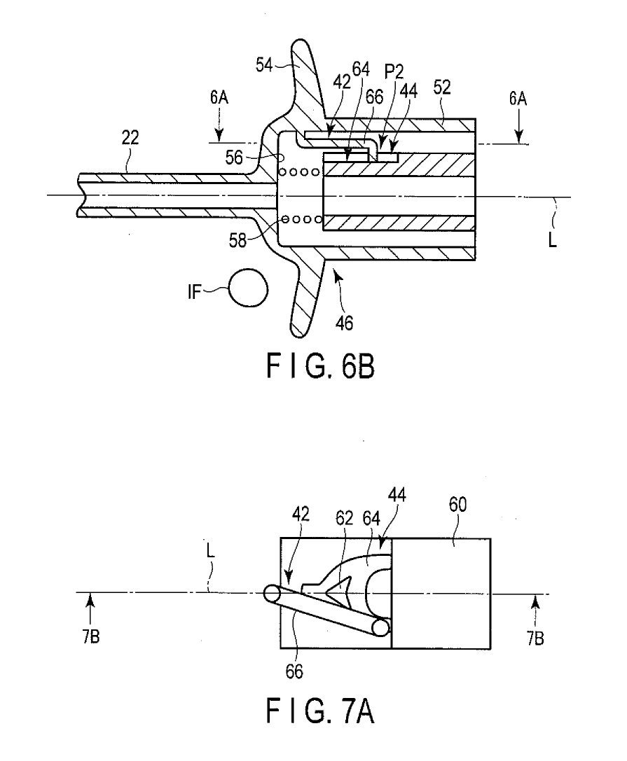

When the engagement pawl 66 reaches the second groove portion 74 from the first groove portion 72, direct return of the engagement pawl 66 to the first groove portion 72 from the second groove portion 74 is regulated by the step 73. As shown in FIG. 6A to FIG. 6C, the engagement pawl 66 is disposed at the lock position P2 by the urging force of the urging member 58 when the operational body 46 is released.

At this point, the distance between the unlock position P1 and the lock position P2 is smaller than a component in the direction along the longitudinal axis L in the distance between the unlock position P1 of the transmitting member 22 and the boundary between the first groove portion 72 and the second groove portion 74. Thus, the rotation amount of the end effector 14 is smaller than the maximum rotation amount state. Since the position of the engagement pawl 66 is regulated, the end effector 14 maintains a certain amount of rotation relative to the distal end of the sheath 12. Thus, this treatment device 10 allows the end effector 14 to be temporarily held rotated relative to the distal end of the sheath 12 by a simple operation.

When the end effector 14 has a pair of openable and closable jaws, the pair of jaws can be relatively opened and closed by an operation to move the open-close knob 36 disposed in the grasp portion 16 closer to or away from the other end 32b of the grasp portion 16. At this point, it is possible to keep grasping the living tissue while the end effector 14 is rotated relative to the distal end of the sheath 12.

When the end effector 14 is located at a position where the end effector 14 is bent relative to the distal end of the sheath 12, the end effector 14 shakes if an external force is applied to the end effector 14. However, unless the extension 54 is moved backward along the longitudinal axis L, a large rotation that may cause the end effector 14 to be straight relative to the sheath 12 is prevented. Since the sheath 12 and the transmitting member 22 have concentric cylindrical shapes in this embodiment, the sheath 12 and the transmitting member 22 are low in flexibility and can maintain high rigidity.

If the extension 54 is rotated around the longitudinal axis L, the transmitting member 22 and the sheath 12 rotate relative to the grasp portion 16 because the engagement pawl 66 of the first locking portion 42 is engaged with the locking groove 64 of the second locking portion 44. Thus, it is possible to suitably adjust the direction of the end effector 14 relative to the grasp portion 16. The living tissue can be easily treated by such a suitable adjustment of the direction of the end effector 14.

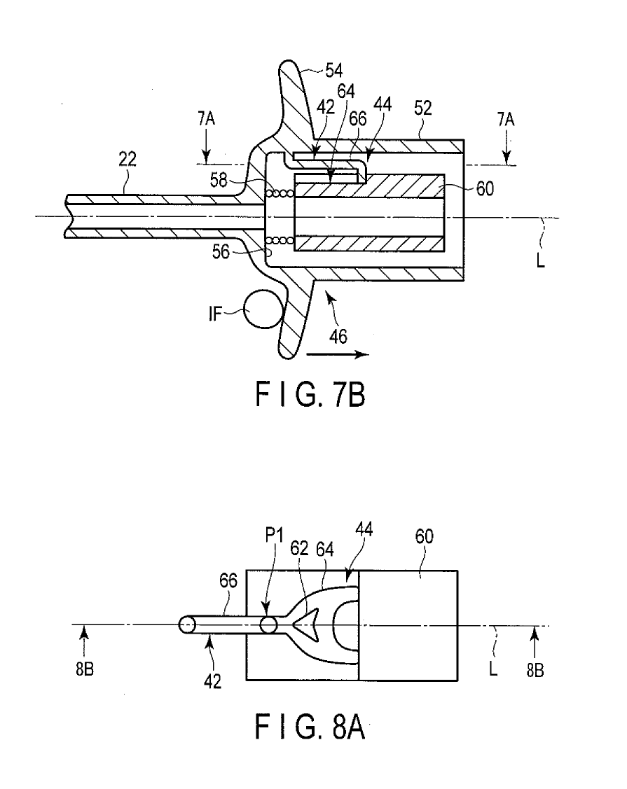

After the living tissue is grasped and suitably treated with the end effector 14, the distal end of the sheath 12 and the end effector 14 are pulled out of the body cavity. Before this work, the living tissue is released. Further work is done so that the end effector 14 becomes straight relative to the distal end of the sheath 12 along the longitudinal axis L. The extension 54 is operated backward along the longitudinal axis L while the engagement pawl 66 of the first locking portion 42 is disposed in the second groove portion 74 (located at the lock position P2). As shown in FIG. 7A and FIG. 7B, the engagement pawl 66 moves toward the boundary between the second groove portion 74 and the third groove portion 76 from the lock position P2. When the first locking portion 42 reaches the third groove portion 76 from the second groove portion 74, direct return of the engagement pawl 66 to the second groove portion 74 from the third groove portion 76 is regulated by the step 75.

The boundary between the second groove portion 74 and the third groove portion 76 is located backward as compared to the lock position P2 along the longitudinal axis L. The transmitting member 22 is pulled backward relative to the sheath 12. Thus, the rotation amount of the end effector 14 temporarily surpasses the lock position P2.

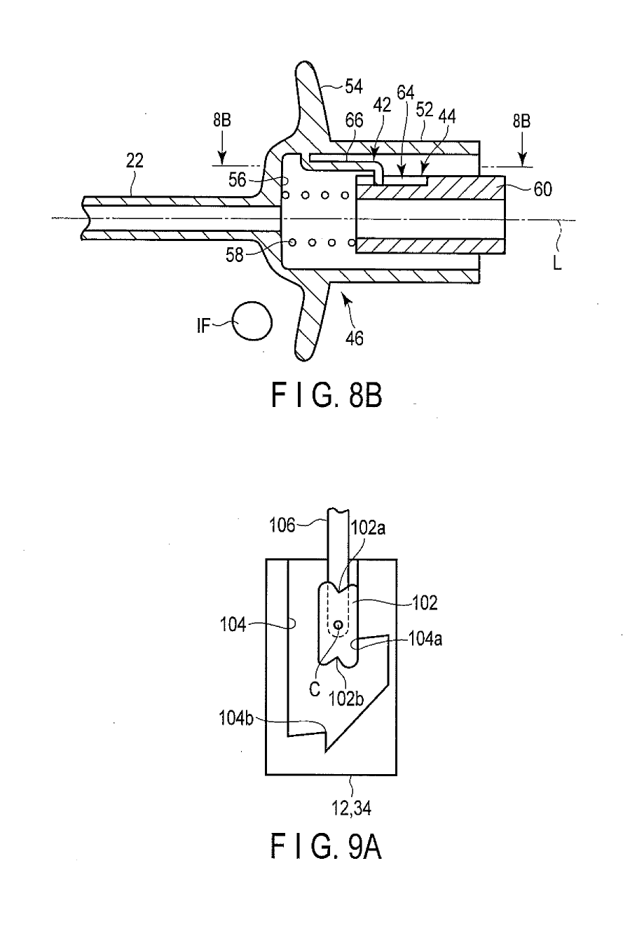

If the extension 54 of the operational body 46 is released, the engagement pawl 66 is disposed at the unlock position P1 of the first groove portion 72 by the urging force of the urging member 58. In this instance, direct return of the engagement pawl 66 to the third groove portion 76 from the first groove portion 72 is regulated by the step 77. Accordingly, the engagement pawl 66 moves to the position of the boundary between the first groove portion 72 and the third groove portion 76 from the position of the boundary between the second groove portion 74 and the third groove portion 76. Therefore, as shown in FIG. 8A and FIG. 8B, the transmitting member 22 moves forward relative to the sheath 12. Thus, the end effector 14 is pushed out on the second rotation shaft S2, and the end effector 14 rotates around the first rotation shaft S1. Consequently, the end effector 14 becomes straight relative to the distal end of the sheath 12 along the longitudinal axis L. In this state, the distal end of the sheath 12 and the end effector 14 are pulled out of the body cavity.

As described above, the following can be said according to the treatment device 10 in this embodiment.

The user can easily switch the end effector 14 at the distal end of the sheath 12 to the position to be straight along the longitudinal axis L and the position to be bent simply by grasping the grasp portion 16 of the treatment device 10 with one hand and then operating the extension 54 of the operational body 46 backward along the longitudinal axis L of the sheath 12 to release the extension 54. Thus, according to the treatment device 10 in this embodiment, it is possible to temporarily hold the end effector 14 rotated relative to the distal end of the sheath 12 by a simple operation.

In this instance, for example, the extension 54 of the operational body 46 is only operated backward along the longitudinal axis L with the index finger while the open-close knob 36 is being held with one of the middle to little fingers. If the extension 54 is released after the extension 54 of the operational body 46 has been operated backward along the longitudinal axis L with the index finger, the transmitting member 22 can be switched by the urging force of the urging member 58 between the position (unlock position) P1 where the transmitting member 22 is moved forward relative to the sheath 12 and the position (lock position) P2 where the transmitting member 22 is moved backward. That is, the treatment device 10 according to this embodiment allows the end effector 14 at the distal end of the sheath 12 to be switched to a suitable position solely by one-hand operation.

The extension 54 of the operational body 46 can be rotated around the longitudinal axis L with the index finger. By this operation, the sheath 12, the end effector 14, and the transmitting member 22 can be suitably rotated around the longitudinal axis L. This allows the end effector 14 to face in a suitable direction. Performing this operation also only requires one hand.

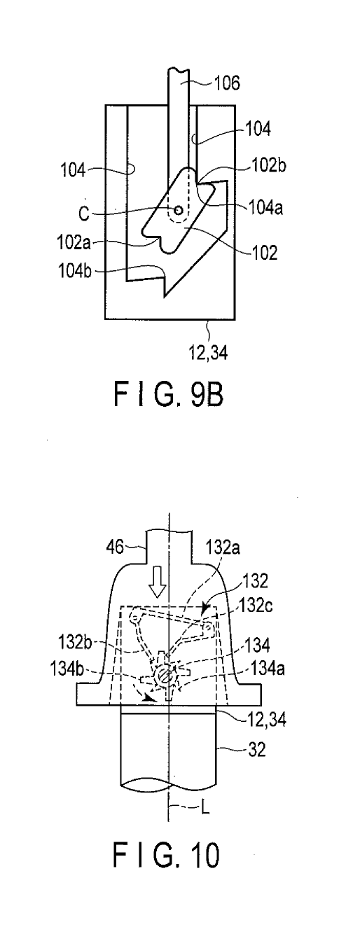

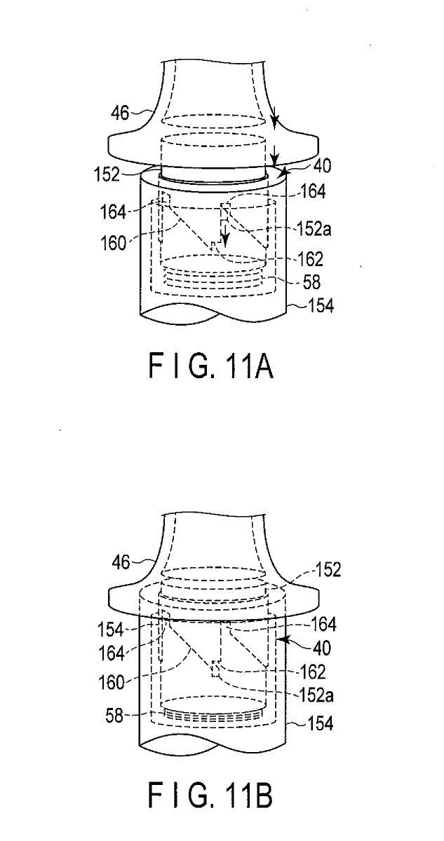

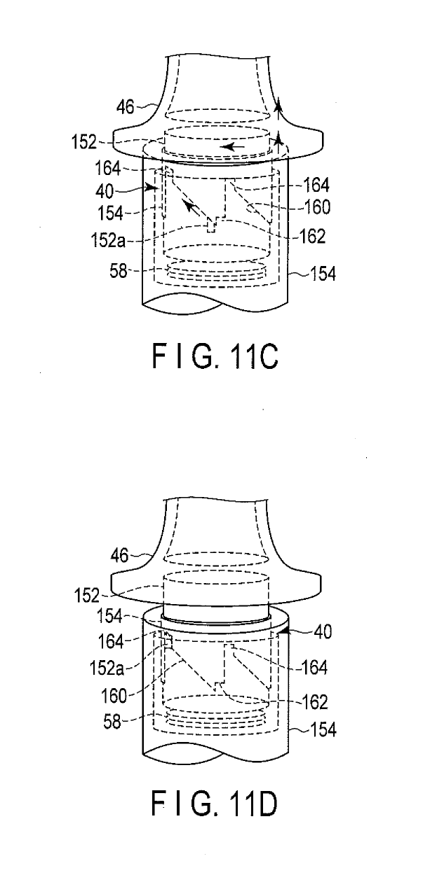

In the example described according to this embodiment, the position holding mechanism 40 using the heart-shaped cam type is used. Instead of using the heart-shaped cam type, the position holding mechanism 40 may use, for example, a rotating cam type shown in FIG. 9A and FIG. 9B, a ratchet cam type shown in FIG. 10, or a rotary cam type shown in FIG. 11A to FIG. 11D.

The position holding mechanism 40 shown in FIG. 9A and FIG. 9B has a cam (first locking portion) 102 which moves along the longitudinal axis L, and a recessed frame (second locking portion) 104 which contacts the circumferential end face of the cam 102.

For example, the cam 102 has a moving rod 106 which moves parallel to the longitudinal axis L in accordance with the motion of the operational body 46. The recessed frame 104 is disposed in the outer circumferential surface of the grasp side cylindrical portion 34 of the grasp portion 16, and is rotatable around the longitudinal axis L, but the movement of the recessed frame 104 in the forward and backward direction is preferably regulated.

The cam 102 has cutouts 102a and 102b at both ends, and has a central shaft C rotatably attached to the moving rod 106. The recessed frame 104 has two separately disposed corners, 104a and 104b, with which the cutouts 102a and 102b engage.

The point of contact between the cam 102 and the recessed frame 104 moves in accordance with the backward movement of the operational body 46, that is, the backward movement of the moving rod 106. The cam 102 holds the active state when the distal-side corner 104a of the two corners 104a and 104b of the recessed frame 104 enters one of the cutouts 102a and 102b of the rotating cam 102. That is, the lock position is set.

If the operational body 46, that is, the moving rod 106 is again moved backward, the corner 104a of the recessed frame 104 comes off one of the cutouts 102a and 102b, and the cam 102 rotates in contact with the proximal-side corner 104b. If the operational force is then removed, the cam 102 returns to a free position as shown in FIG. 9A. That is, the unlock position is set.

Therefore, the user can temporarily hold the end effector 14 rotated relative to the distal end of the sheath (insertion portion) 12 by a simple operation of the operational body 46.

As shown in FIG. 10, the position holding mechanism 40 has a pawl (first locking portion) 132 having both its ends supported, and a cam (second locking portion) 134. The pawl 132 is supported on the operational body 46, and the cam 134 is disposed, for example, in the outer circumferential surface of the grasp side cylindrical portion 34 of the grasp portion 16 or in the outer circumferential surface of the proximal end of the sheath 12. The pawl 132 has an input portion 132a which is activated by the operational body 46, a feed pawl 132b which rotates the cam 134 together with the input portion 132a, and a stop pawl 132c which stops the rotation of the cam 134 that has been rotated by a predetermined amount together with the input portion 132a. The cam 134 has a feed pawl 134a which is rotated a predetermined amount by the feed pawl 132b, and a stop pawl 134b which stops the rotation of the cam 134 that has been rotated by a predetermined amount by the stop pawl 132c.

The operational force is transmitted to the feed pawl 132b via the input portion 132a by the operational body 46 located at the front position along the longitudinal axis L shown in FIG. 10. The cam 134 rotates in a direction indicated by an arrow in FIG. 10. However, one operation of the operational body 46 alone does not rotate the cam 134 by a certain amount or more due to the stop pawl 132c. If the operational body 46 is again operated to push the feed pawl 132b, the cam 134 again rotates. Thus, the operational body 46 is movable within a predetermined range along the longitudinal axis L. This operation is repeated so that the operational body 46 is switched between the unlock position and the lock position. Therefore, the end effector 14 moves as shown in FIG. 1A.

Therefore, the user can temporarily hold the end effector 14 rotated relative to the distal end of the sheath (insertion portion) 12 by a simple operation of the operational body 46.

As shown in FIG. 11A to FIG. 11D, the position holding mechanism (alternate mechanism) 40 has a movable body (first locking portion) 152 having, for example, a cylindrical shape, and a tubular body (second locking portion) 154 disposed on the outer circumference of the movable body 152. The movable body 152 is provided on the proximal side of the transmitting member 22. The tubular body (second locking portion) 154 is provided in the outer circumferential surface of the grasp side cylindrical portion 34 or in the outer circumferential surface of the proximal end of the sheath 12.

The movable body 152 is urged upward (toward the end effector 14) in FIG. 11A to FIG. 11D by the urging member 58. The movable body 152 has, in its outer circumferential surface, a protrusion 152a. The tubular body 154 has, in its inner circumferential surface, cutouts 162 and 164 in a back end side portion and a root portion (distal end side portion) of a saw-tooth-shaped angle portion 160.

In FIG. 11A, the operational body 46 is at the unlock position (the position where the end effector 14 extends along the longitudinal axis L of the sheath 12). As shown in FIG. 11A, the movable body 152 is urged upward in FIG. 11A by the urging force of the urging member 58, and the protrusion 152a of the movable body 152 engages with the second cutout 164 formed in the root portion of the angle portion 160 in the inner circumferential surface of the tubular body 154. That is, the movable body 152 is pushed upward (closer to the end effector 14) by the urging force of the urging member 58 applied to the inner circumferential surface (bottom surface) of the tubular body 154, and the protrusion 152a of the movable body 152 engages with the second cutout 164 formed in the root portion of the angle portion 160 of the tubular body 154.

If the operational body 46 located at the position shown in FIG. 11A is then pushed toward the back end along the longitudinal axis L, the movable body 152 is pushed by the operational body 46 and thus contracts the urging member 58. If the force to push the operational body 46 is loosened in this state, the movable body 152 is slightly lifted upward by the urging force of the urging member 58. In this instance, the protrusion 152a of the movable body 152 engages with the first cutout 162 formed in the distal portion of the angle portion 160 of the tubular body 154. Thus, the movable body 152 shown in FIG. 11B is kept moved toward the back end as compared to the movable body 152 shown in FIG. 11A.

If the operational body 46 in the state shown in FIG. 1B is again pushed, the movable body 152 contracts the urging member 58 and is then slightly pushed down, so that the protrusion 152a of the movable body 152 which has engaged with the first cutout 162 of the tubular body 154 comes off the first cutout 162. If the force to push the operational body 46 is loosened in this state, the movable body 152 is lifted upward by the urging force of the urging member 58. Accordingly, the movable body 152 moves relative to the tubular body 154 from the state shown in FIG. 11C to the state shown in FIG. 11D.

Therefore, the user can temporarily hold the end effector 14 rotated relative to the distal end of the sheath (insertion portion) 12 by a simple operation of the operational body 46.

Moreover, the movable body (first locking portion) 152 rotates relative to the tubular body 154. In this instance, an unshown support such as a ball bearing is disposed between the operational body 46 and the movable body 152, and prevents the transmitting member 22 from rotating when the movable body 152 rotates relative to the tubular body 154. The operational body 46 and the transmitting member 22 are supported by a suitable frictional force to prevent the operational body 46 and the transmitting member 22 from being automatically rotated by, for example, gravitation. The transmitting member 22 is rotated around the longitudinal axis L by the operation of the operational body 46.

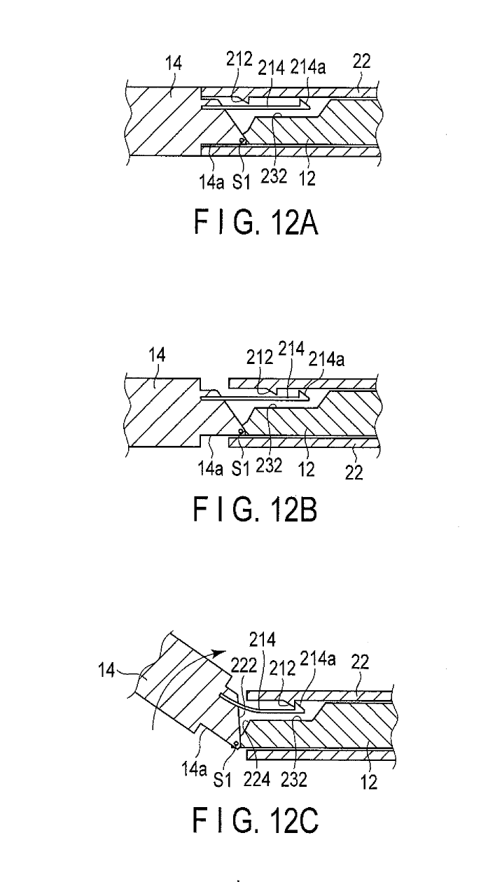

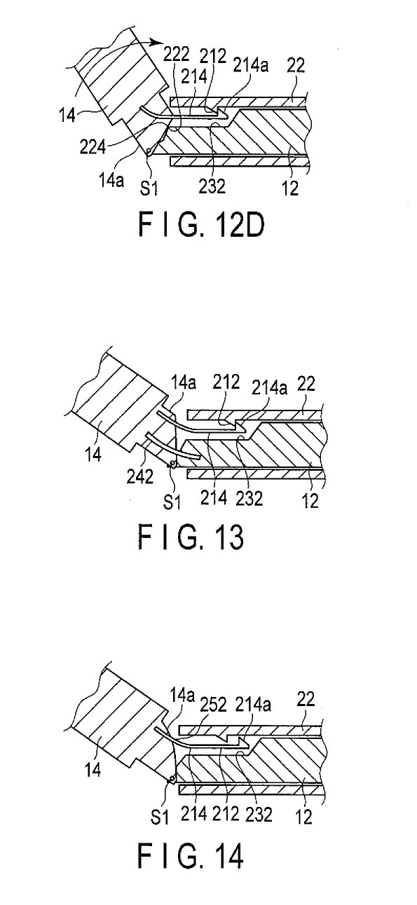

Next, the second embodiment is described with reference to FIG. 12A to FIG. 12D. This embodiment is a modification of the first embodiment, and the same components or the components having the same functions as those described in the first embodiment are provided with the same reference marks and are not described in detail.

The structure described in this embodiment increases the rigidity to keep the end effector 14 straight when the end effector 14 is straight relative to the distal end of the sheath 12.

The user can temporarily hold the end effector 14 rotated relative to the distal end of the sheath (insertion portion) 12 by a simple operation of the operational body 46.

In this embodiment, instead of the second rotation shaft S2, a first locking body 212 is disposed in the inner circumferential surface of the distal end of the transmitting member 22, and a second locking body 214 is disposed in the end effector 14. The first locking body 212 is formed as an engagement protrusion in the inner circumferential surface of the distal end of the transmitting member 22. The second locking body 214 is formed as a belt-shaped body having, at its proximal end, an engagement protrusion 214a which is switched between a state to be caught on the first locking body 212 (caught state) and a state to be released from the caught state (non-caught state). The second locking body 214 has a proper level of flexibility. The second locking body 214 may be suitably expandable in its longitudinal direction.

As shown in FIG. 12A to FIG. 12D, a recess (fit portion) 14a into which the distal end of the transmitting member (outer tube) 22 is fitted is formed at the proximal end of the end effector 14. The recess (fit portion) 14a is formed by removing the outer circumference of the proximal end of the end effector 14 in a tubular shape so that the distal end (fit portion) of the transmitting member 22 is fitted in an abutment state as shown in FIG. 12A.

Owing to the position holding mechanism 40, the distal end (fit portion) of the transmitting member 22 is fitted into the recess (fit portion) 14a at the proximal end of the end effector 14 as shown in FIG. 12A when the transmitting member 22 is located forward relative to the sheath 12. Thus, when the transmitting member 22 is located forward relative to the sheath 12, the end effector 14 is straight along the longitudinal axis L.

In contrast, when the transmitting member 22 is located backward relative to the sheath 12, the distal end of the transmitting member 22 is unfitted from the recess 14a at the proximal end of the end effector 14 as shown in FIG. 12B to FIG. 12D. Thus, the second locking body 214 is caught on the first locking body 212, and the end effector 14 is rotated to deviate from the longitudinal axis L.

The first locking body 212 and the second locking body 214 are engaged and disengaged when the engagement pawl 66 of the position holding mechanism 40 moves backward further than the lock position P2 along the longitudinal axis L from the unlock position P1 and then moves forward.

The proximal end of the end effector 14 has a first abutment surface (end effector side abutment surface) 222 which abuts on the distal end of the sheath 12. The distal end of the sheath 12 has a second abutment surface (sheath side abutment surface) 224 which abuts on the first abutment surface 222 at the proximal end of the end effector 14. If the first abutment surface 222 and the second abutment surface 224 are formed into suitable shapes and their abutment positions are adjusted, the rotation angle of the end effector 14 to the distal end of the sheath 12 can be adjusted.

When the end effector 14 is straight relative to the distal end of the sheath 12, the recess 14a at the proximal end of the end effector 14 is disposed inside the distal end of the transmitting member 22. Thus, when an external force is applied to the end effector 14 from the direction that deviates from the longitudinal axis L, the rotation of the end effector 14 relative to the distal end of the sheath 12 can be inhibited by the distal end of the transmitting member 22.

When the end effector 14 is straight relative to the distal end of the sheath 12, the first locking body 212 is located closer to the proximal side than the proximal end of the end effector 14 while the recess 14a at the proximal end of the end effector 14 is disposed inside the distal end of the transmitting member 22. Thus, it is possible to prevent the first locking body 212 from interfering with the end effector 14.

In contrast, when the end effector 14 is bent relative to the distal end of the sheath 12, the distal end of the transmitting member 22 moves backward relative to the distal end of the sheath 12 to expose the circular recess 14a at the proximal end of the end effector 14.

A guide portion 232 which moves the first locking body 212 and the second locking body 214 of the transmitting member 22 are provided at the distal end of the sheath 12. This guide portion 232 prevents the second locking body 214 from interfering with the distal end of the sheath 12 and the inner circumferential surface of the distal end of the transmitting member 22 when the transmitting member 22 is moved as shown in FIG. 12A to FIG. 12D.

Next, a first modification of the second embodiment is described with reference to FIG. 13. This modification is a modification of the second embodiment, and the same components or components having the same functions as those described in the first and second embodiments are provided with the same reference marks and are not described in detail.

The user can temporarily hold the end effector 14 rotated relative to the distal end of the sheath (insertion portion) 12 by a simple operation of the operational body 46.

As shown in FIG. 13, an urging body 242 which urges the end effector 14 to rotate relative to the distal end of the sheath 12 is disposed between the distal end of the sheath 12 and the proximal end of the end effector 14. Thus, the end effector 14 can be easily rotated relative to the distal end of the sheath 12 by the urging force of the urging body 242 in addition to the tensile force of the second locking body 214. In the meantime, while the recess 14a at the proximal end of the end effector 14 is fitted in the inner circumferential surface of the distal end of the cylindrical transmitting member 22, the end effector 14 is kept straight relative to the distal end of the sheath 12. This prevents the state in which the end effector 14 is kept straight relative to the distal end of the sheath 12 from being affected even if the urging body 242 extends.

Next, a second modification of the second embodiment is described with reference to FIG. 14. This modification is a modification of the second embodiment, and the same components or components having the same functions as those described in the first embodiment and the second embodiment including the first modification are provided with the same reference marks and are not described in detail.

The user can temporarily hold the end effector 14 rotated relative to the distal end of the sheath (insertion portion) 12 by a simple operation of the operational body 46.

A taper portion 252 in which the first abutment surface 222 is tapered is provided. Thus, the inner circumferential surface of the distal end of the transmitting member 22 can be easily guided to the recess 14a of the end effector 14 by the taper portion 252, and the end effector 14 can be easily moved when the end effector 14 is disposed straight along the longitudinal axis L relative to the distal end of the sheath 12.

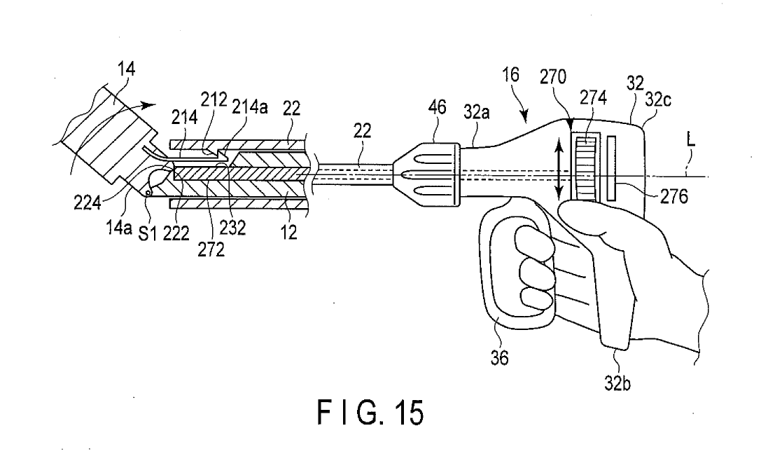

Next, a third modification of the second embodiment is described with reference to FIG. 15. This modification is a modification of the first and second modifications of the second embodiment, and the same components or components having the same functions as those described in the first embodiment and the second embodiment including the first and second modifications are provided with the same reference marks and are not described in detail.

The user can temporarily hold the end effector 14 rotated relative to the distal end of the sheath (insertion portion) 12 by a simple operation of the operational body 46.

In this modification, an adjustment mechanism 270 which adjusts the amount of rotation of the end effector 14 relative to the distal end of the sheath 12 is described. The adjustment mechanism 270 can adjust a rotation angle at which the end effector 14 around the first rotation shaft S1 is kept in the second state where the end effector 14 is rotated relative to the longitudinal axis L at the distal end of the sheath 12.

The first abutment surface 222 at the proximal end of the end effector 14 is preferably disposed on the longitudinal axis (central axis) L either in the state in which the end effector 14 is straight or in the state in which the end effector 14 is rotated relative to the distal end of the sheath 12.

In this embodiment, an abutment rod (moving body) 272 which is movable in the direction of the longitudinal axis (central axis) L is inserted on the longitudinal axis L inside the sheath 12. The distal end of the abutment rod 272 can be in and out of contact with the first abutment surface 222. The proximal end of the abutment rod 272 is coupled to an adjustment knob (rotation amount adjustment body) 274 disposed in the grasp portion 16 via an unshown ball screw. The adjustment knob 274 is disposed in the grasp portion 16 rotatably around the longitudinal direction of the abutment rod 272. The grasp portion 16 exposes part of the adjustment knob 274 to the outside. The abutment rod 272 and the adjustment knob 274 constitute the adjustment mechanism 270.

An index 276 indicating the maximum rotation amount of the end effector 14 is preferably disposed in the vicinity of the adjustment knob 274 of the main body 32 of the grasp portion 16.

If the adjustment knob 274 is suitably rotated, the abutment rod 272 moves forward and backward along the longitudinal axis L owing to the effects of the ball screw. The adjustment knob 274 moves the abutment rod 272 along its axial direction and adjusts the position where the abutment rod 272 abuts on the end effector 14 to adjust the amount of rotation of the end effector 14 relative to the distal end of the sheath 12. The maximum rotation amount is reduced if the abutment rod 272 is moved forward. The maximum rotation amount is increased if the abutment rod 272 is moved backward. At this point, it is possible to check the maximum rotation amount of the end effector 14 relative to the longitudinal axis L of the sheath 12, for example, solely by visual recognition.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

1. A treatment device comprising:
an insertion portion which includes a distal end, a proximal end, and a longitudinal axis defined by the distal end and the proximal end;
an end effector which is rotatably provided at the distal end of the insertion portion;
a transmitting member which moves back and forth along a longitudinal axis of the insertion portion to transmit a drive force to rotate the end effector;
a first locking portion which is provided on the proximal side of the transmitting member;
a second locking portion which is provided on the proximal side of the insertion portion and which is configured to engage with the first locking portion;
an operational body which is provided on the proximal side of the transmitting member, which activates the first locking portion relative to the second locking portion, which is configured to switch between a first position where the transmitting member is moved forward along the longitudinal axis of the insertion portion to keep the end effector in a first state to extend along the longitudinal axis relative to the distal end of the insertion portion along the longitudinal axis, and a second position where the transmitting member is moved backward along the longitudinal axis of the insertion portion to keep the end effector in a second state to be rotated relative to the longitudinal axis with respect to the distal end of the insertion portion;
an urging body which is disposed between the second locking portion and the operational body and which urges the operational body forward along the longitudinal axis of the insertion portion.

2. The treatment device according to claim 1, wherein the first and second locking portions and the operational body form a position holding mechanism by which the first and second locking portions hold positions relative to each other.

3. The treatment device according to claim 2, wherein the first and second locking portions of the position holding mechanism are one of a heart cam type, a rotating cam type, a ratchet cam type, and a rotary cam type.

4. (canceled)

5. (canceled)

6. The treatment device according to claim 1, wherein the first locking portion, the second locking portion, and the operational body are rotatable around the longitudinal axis.

7. The treatment device according to claim 1, wherein
the insertion portion is formed as an inner tube, and
the transmitting member is formed as an outer tube which is movable relative to the inner tube along the longitudinal axis.

8. The treatment device according to claim 7, comprising a flexible locking body which is disposed in the end effector and which is configured to engage with an engagement protrusion provided in an inner circumferential surface of the transmitting member, the locking body being switched between a state in which the locking body is caught on the engagement protrusion and a state in which the locking body is not caught on the engagement protrusion in accordance with the movement of the transmitting member.

9. The treatment device according to claim 7, wherein
the insertion portion includes a first rotation shaft which intersects at right angles with the longitudinal axis and which serves as a supporting point of the rotation of the end effector, and
the operational body includes an adjustment mechanism configured to adjust a rotation angle at which the end effector around the first rotation shaft is rotated relative to the longitudinal axis at the distal end of the insertion portion and kept in the second state.

10. The treatment device according to claim 9,
comprising a grasp portion provided at the proximal end of the insertion portion and grasped by a user;
wherein
the adjustment mechanism includes:
a moving body which is disposed parallel to the longitudinal axis and which abuts on the end effector; and
a rotation amount adjustment body which is provided in the grasp portion and which moves the moving body along its axial direction and adjusts the position where the moving body abuts on the end effector to adjust the amount of rotation of the end effector relative to the distal end of the insertion portion.

11. The treatment device according to claim 1, wherein the transmitting member is cylindrical, and is movable between a front position where the transmitting member moves forward relative to the insertion portion along the longitudinal axis and is fitted into the outer circumferential surface of the proximal end of the end effector so that the end effector is in a first state to extend along the longitudinal axis relative to the distal end of the insertion portion, and a back position where the transmitting member moves backward relative to the insertion portion along the longitudinal axis and is unfitted from the outer circumferential surface of the proximal end of the end effector so that the end effector is in the second state relative to the distal end of the insertion portion.

12. The treatment device according to claim 11, wherein the end effector includes a fit portion which is fitted into the distal end of the transmitting member in the first state.

13. (canceled)

14. (canceled)

15. A treatment device comprising:
an insertion portion which includes a distal end, a proximal end, and a longitudinal axis defined by the distal end and the proximal end;
an end effector which is rotatably provided at the distal end of the insertion portion;
a transmitting member which transmits a drive force to rotate the end effector;
a first locking portion which is provided on the proximal side of the transmitting member;
a second locking portion which is provided on the proximal side of the insertion portion and which is configured to engage with the first locking portion;
an operational body which is provided on the proximal side of the transmitting member, which activates the first locking portion relative to the second locking portion, which is configured to switch between a first position where the transmitting member is moved forward along the longitudinal axis of the insertion portion to keep the end effector in a first state to extend along the longitudinal axis relative to the distal end of the insertion portion along the longitudinal axis, and a second position where the transmitting member is moved backward along the longitudinal axis of the insertion portion to keep the end effector in a second state to be rotated relative to the longitudinal axis with respect to the distal end of the insertion portion; and
an urging body which is disposed between the second locking portion and the transmitting member and which urges the transmitting member forward along the longitudinal axis of the insertion portion.

16. A treatment device comprising:
an insertion portion which includes a distal end, a proximal end, and a longitudinal axis defined by the distal end and the proximal end;
an end effector which is rotatably provided at the distal end of the insertion portion;
a transmitting member which transmits a drive force to rotate the end effector;
a first locking portion which is provided on the proximal side of the transmitting member;
a second locking portion which is provided on the proximal side of the insertion portion and which is configured to engage with the first locking portion;
an operational body which is provided on the proximal side of the transmitting member, which activates the first locking portion relative to the second locking portion, which is configured to switch between a first position where the transmitting member is moved forward along the longitudinal axis of the insertion portion to keep the end effector in a first state to extend along the longitudinal axis relative to the distal end of the insertion portion along the longitudinal axis, and a second position where the transmitting member is moved backward along the longitudinal axis of the insertion portion to keep the end effector in a second state to be rotated relative to the longitudinal axis with respect to the distal end of the insertion portion; and
an urging body which is disposed between the second locking portion and the operational body and which urges the operational body forward along the longitudinal axis of the insertion portion.
